(19) United States
(12) Patent Application Publication
(10) Pub. No.: US 2024/0339194 A1
(43) Pub. Date: Oct. 10, 2024

(54) MEDICAL ASSISTANCE SYSTEM, MEDICAL ASSISTANCE METHOD, AND STORAGE MEDIUM

(71) Applicant: OLYMPUS MEDICAL SYSTEMS CORP., Tokyo (JP)

(72) Inventors: Yusuke KATO, Tokyo (JP); Kazuyoshi TAMURA, Tokyo (JP); Yuta KOBAYASHI, Tokyo (JP); Tatsuki ODEISHI, Tokyo (JP)

(73) Assignee: OLYMPUS MEDICAL SYSTEMS CORP., Tokyo (JP)

(21) Appl. No.: 18/745,187

(22) Filed: Jun. 17, 2024

Related U.S. Application Data
(63) Continuation of application No. PCT/JP2021/047090, filed on Dec. 20, 2021.

(51) Int. Cl. G16H 20/40; G16H 10/60; G16H 15/00; G16H 30/40; G16H 50/70

(52) U.S. Cl. CPC G16H 20/40; G16H 10/60; G16H 15/00; G16H 30/40; G16H 50/70

(57) ABSTRACT

A lesion identification information acquisition unit acquires lesion identification information including the result of identifying a lesion confirmed in a current endoscopy of a patient being examined. An examination history information acquisition unit acquires examination history information including a diagnosis history for a past endoscopic examination of the patient being examined. A recommended action information output unit compares the lesion identification information and the examination history information with a prescribed determination criterion (created on the basis of, e.g., accumulated data from past endoscopic examinations) and outputs recommended action information including a recommended action for the lesion confirmed in the current endoscopic examination.

### CROSS-REFERENCE TO RELATED APPLICATIONS

This application is based upon and claims the benefit of priority from the International Application No. PCT/JP2021/047090, filed on Dec. 20, 2021, the entire contents of which are incorporated.

### BACKGROUND

### 1. Technical Field

The present disclosure relates to medical assistance systems, medical assistance methods, and storage media for assisting doctors in determining actions to be performed during endoscopic examinations or the like.

### 2. Description of the Related Art

In the related art, medical diagnosis support systems have been known that have a differentiation function that classifies the type of lesion using a computer at the time of endoscopic observation. If there are actions to take for a lesion and the details of the actions are left to the judgment of the doctor, and even if the lesion is discovered using the medical diagnosis support system, there is a risk that the lesion will not be treated appropriately and left untreated due to the doctor's error in judgment. When determining if there are actions to take for a lesion and the details of the actions, it is desirable to estimate the progression of the lesion while taking into account not only the type of lesion but also a past examination history so as to make a decision.

Since conventional medical diagnosis support systems only show the classification results of the type of lesion, there is room for improvement as a support for determining if there are actions to take for a lesion and the details of the actions.

### SUMMARY

The present disclosure addresses the above-described issue, and a purpose thereof is to provide a technology that can assist in determining if there are actions to take for a lesion and the details of the actions.

A medical assistance system according to one embodiment of the present disclosure is a medical assistance system including: at least one processor including hardware. The at least one processor is configured to: acquire lesion identification information including the result of identifying a lesion confirmed in a current endoscopic examination of a patient being examined; acquire examination history information including a diagnosis history for a past endoscopic examination of the patient being examined; and compare the lesion identification information and the examination history information with a predetermined determination criterion and output recommended action information including a recommended action for the lesion confirmed in the current endoscopic examination.

Another embodiment of the present disclosure relates to a medical assistance method. This method is a medical assistance method including: acquiring lesion identification information including the result of identifying a lesion confirmed in a current endoscopic examination of a patient being examined; acquiring examination history information including a diagnosis history for a past endoscopic examination of the patient being examined; and comparing the lesion identification information and the examination history information with a predetermined determination criterion and outputting recommended action information including a recommended action for the lesion confirmed in the current endoscopic examination.

Optional combinations of the aforementioned constituting elements and implementations of the present disclosure in the form of methods, apparatuses, systems, recording mediums, and computer programs may also be practiced as additional modes of the present disclosure.

### BRIEF DESCRIPTION OF THE DRAWINGS

Embodiments will now be described, by way of example only, with reference to the accompanying drawings which are meant to be exemplary, not limiting, and wherein like elements are numbered alike in several Figures, in which:

FIG. 1 is a diagram showing a configuration example of a medical assistance system according to an embodiment;

FIG. 2 is a diagram showing a configuration example of an endoscope work assistance system of FIG. 1;

FIG. 3A to FIG. 3C are diagrams for explaining a flow until a recommended action according to Specific Example 1 is determined;

FIG. 4 is a diagram showing an endoscopic image of a subject patient displayed on a display device of an endoscope system according to Specific Example 1;

FIG. 5A to FIG. 5C are diagrams for explaining a flow until a recommended action according to Specific Example 2 is determined;

FIG. 6 is a diagram showing an endoscopic image of a subject patient displayed on a display device of an endoscope system according to Specific Example 2;

FIG. 7A to FIG. 7C are diagrams for explaining a flow until a recommended action according to Specific Example 3 is determined;

FIG. 8 is a diagram showing an endoscopic image of a subject patient displayed on a display device of an endoscope system according to Specific Example 3;

FIG. 9 is a flowchart showing the basic operation 5 of the endoscope work assistance system according to the embodiment; and

FIG. 10 is a diagram showing an example of a report input screen.

### DETAILED DESCRIPTION

The disclosure will now be described by reference to the preferred embodiments. This does not intend to limit the scope of the present disclosure, but to exemplify the disclosure.

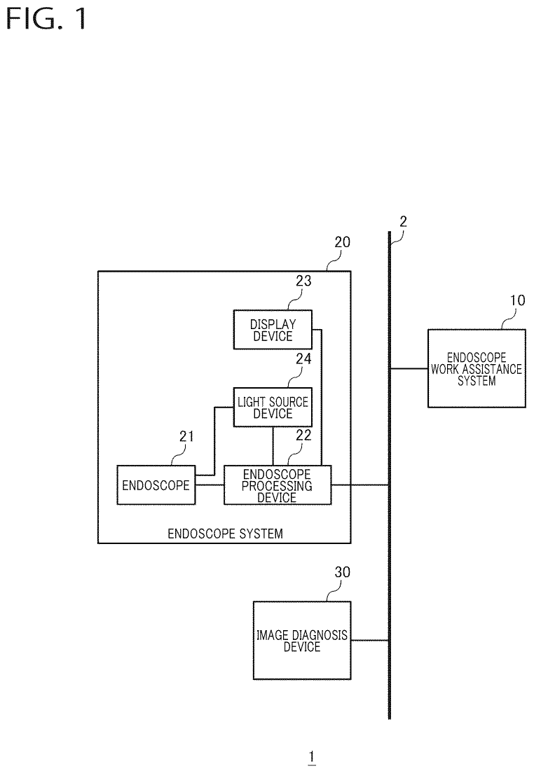

FIG. 1 shows a configuration example of a medical assistance system according to an embodiment. The medical assistance system 1 according to the embodiment is a system for assisting endoscopic examinations provided in an endoscope department of a medical facility. The medical assistance system 1 includes an endoscope work assistance system 10, an endoscope system 20, and an image diagnosis device 30. The endoscope work assistance system 10, the endoscope system 20, and the image diagnosis device 30 are communicatively connected by a network 2 such as a LAN.

The endoscope system 20 includes an endoscope 21, an endoscope processing device (also referred to as a video processor) 22, a display device 23, and a light source device 24. The endoscope 21 is inserted into the patient's body and images the inside of the patient's body.

The endoscope 21 includes a solid-state imaging device. The solid-state imaging device includes a CMOS image sensor, a CCD image sensor, or a CMD image sensor and converts incident light into an electrical signal. The converted image signal (electrical signal) is subjected to signal processing such as A/D conversion and noise removal by a signal processing circuit (not shown), and is output to the endoscope processing device 22. The endoscope 21 includes a forceps channel. The doctor can perform various actions during an endoscopic examination by passing a treatment tool through the forceps channel.

The display device 23 includes a liquid crystal monitor or an organic EL monitor, and displays an image input from the endoscope processing device 22. The display device 23 can display an image of the inside of the patient's body captured by the endoscope 21 as an endoscopic image in real time. The light source device 24 a light source such as a xenon lamp, and supplies observation light (white light, narrow band light, fluorescence, near-infrared light, etc.) to the distal end of the endoscope 21. The light source device 24 also incorporates a pump that sends water and air to the endoscope 21.

The endoscope processing device 22 controls the entire endoscope system 20 in an integrated manner. The endoscope processing device 22 causes the display device 23 to display an endoscope image input from the endoscope 21. At that time, the endoscope processing device 22 can perform various effect processing such as superposition of an on-screen display (OSD) and emphasis on the endoscope image input from the endoscope 21.

The image diagnosis device 30 is a device for detecting lesions through image recognition from an endoscopic image. The image diagnosis device 30 may be a dedicated device, or may be configured as a general-purpose server or a PC on which an image diagnosis program is implemented.

The image diagnosis device 30 has a machine learning model for detecting lesions from an endoscopic image. The machine learning model is generated by machine learning using a large number of endoscopic images annotated with lesions as a supervised dataset. Annotations are given by annotators with specialized knowledge such as doctors. For machine learning, CNNs, RNNs, LSTM, etc., which are types of deep learning, can be used. The image diagnosis device 30 identifies a lesion by applying an endoscopic image input from the endoscope system 20 to a machine learning model, and outputs lesion identification information including the result of identifying the lesion to the endoscope work assistance system 10.

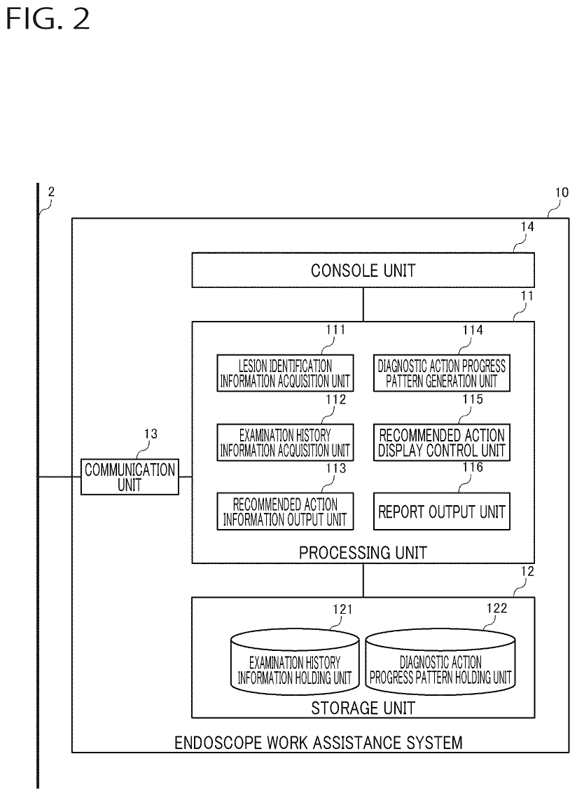

FIG. 2 shows a configuration example of the endoscope work assistance system 10 of FIG. 1. The endoscope work assistance system 10 is constructed with at least one server. The endoscope work assistance system 10 includes a processing unit 11, a storage unit 12, a communication unit 13, and a console unit 14. The processing unit 11 includes a lesion identification information acquisition unit 111, an examination history information acquisition unit 112, a recommended action information output unit 113, a diagnostic action progress pattern generation unit 114, a recommended action display control unit 115, and a report output unit 116.

The configuration of the processing unit 11 is implemented by hardware such as an arbitrary processor (for example, CPU and GPU), memory (for example, DRAM), or other LSIs (for example, FPGA, ASIC), and by software such as a program or the like loaded into the memory. The figure depicts functional blocks implemented by the cooperation of hardware and software. Thus, a person skilled in the art should appreciate that there are many ways of accomplishing these functional blocks in various forms in accordance with the components of hardware only, software only, or the combination of both.

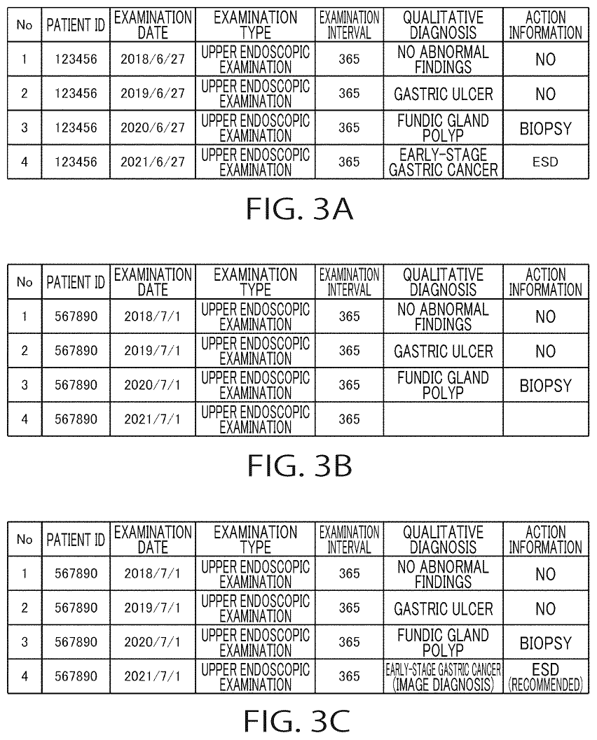

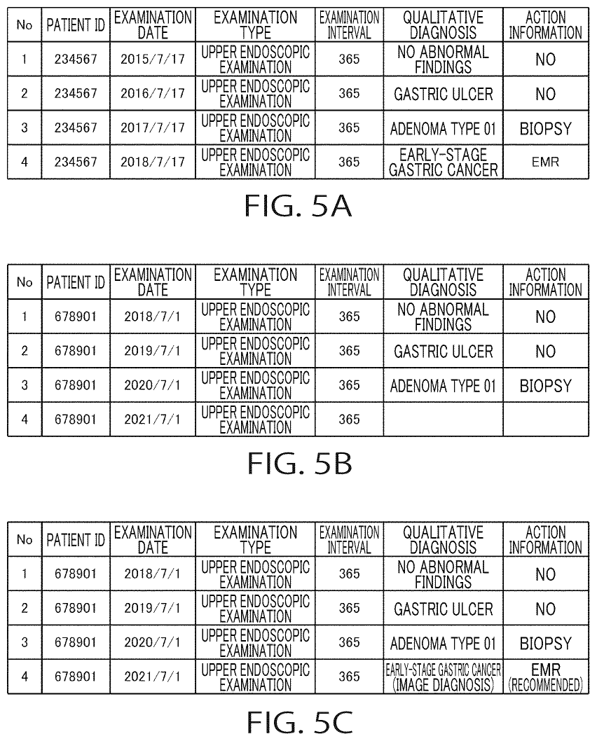

The storage unit 12 includes a storage medium such as an HDD and SSD, and includes an examination history information holding unit 121 and a diagnostic action progress pattern holding unit 122. The examination history information holding unit 121 is a database that accumulates examination history information for each patient. For example, the examination history information includes examination number, patient ID, examination date, examination type, examination interval, qualitative diagnosis, action information, and the like (see FIGS. 3A and 5A).

The qualitative diagnosis includes diagnosis information of a lesion confirmed by the doctor in past endoscopic examinations. If no lesions are found, the qualitative diagnosis is recorded as “no abnormal findings.” When the lesion identified by the image diagnosis device 30 is adopted by the doctor, the lesion identified by the image diagnosis device 30 is directly recorded for the qualitative diagnosis. When the doctor changes the lesion identified by the image diagnosis device 30, the lesion changed by the doctor is recorded for the qualitative diagnosis. In that case, the lesion identified by the image diagnosis device 30 is recorded separately as AI image diagnosis.

The action information includes the action details performed on the diagnosed lesion. If no action is performed, “no action information” is recorded.

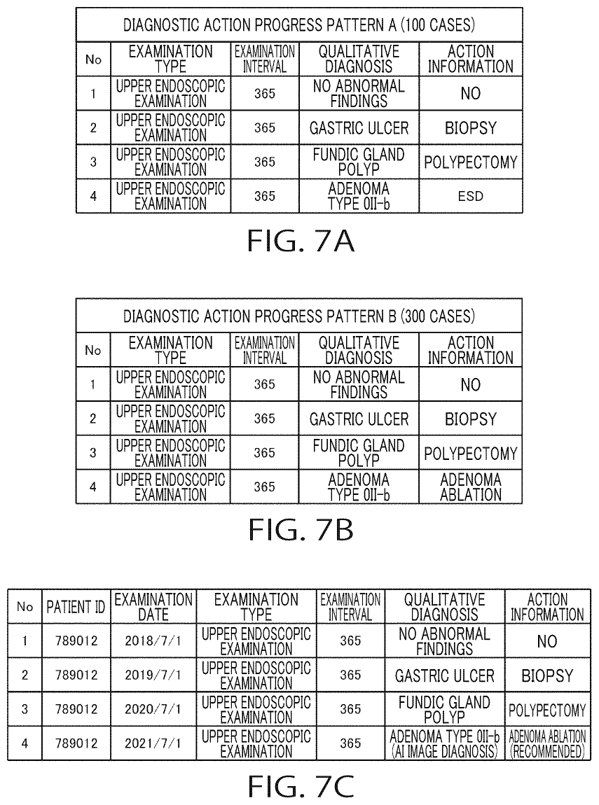

The diagnostic action progress pattern holding unit 122 is a database that uses a plurality of pieces of examination history information accumulated in the examination history information holding unit 121 as reference data and that accumulates a plurality of pieces of diagnostic action progress pattern information created by aggregating pieces of examination history information with a matching examination type, a matching or similar examination interval, matching or similar qualitative diagnosis, and matching action information. For example, the diagnostic action progress pattern information includes examination number, examination type, examination interval, qualitative diagnosis, action information, number of cases, and the like (see FIGS. 7A and 7B).

The communication unit 13 performs a communication process for communicating with the endoscope system 20 or the image diagnosis device 30 via the network 2. The console unit 14 is a user interface including a liquid crystal monitor, an organic EL monitor, a mouse, a keyboard, a touch panel, and the like. The function of the console unit 14 can be replaced by a client PC (not shown) connected via the network 2.

In the current endoscopic examination (hereinafter referred to as “current examination”) of the patient being examined (hereinafter referred to as “subject patient”), the image diagnosis device 30 outputs lesion identification information including the result of identifying the lesion detected through image recognition of an endoscopic image captured by the endoscope system 20 to the endoscope work assistance system 10. The lesion identification information acquisition unit 111 of the endoscope work assistance system 10 acquires lesion identification information from the image diagnosis device 30.

The examination history information acquisition unit 112 acquires examination history information including the diagnosis history for the subject patient's past endoscopic examinations (hereinafter referred to as “past examinations”) from the examination history information holding unit 121. Specifically, the examination history information acquisition unit 112 searches the examination history information holding unit 121 using the patient ID of the subject patient as a key and extracts the examination history information of the subject patient.

The recommended action information output unit 113 compares the lesion identification information of the current examination acquired by the lesion identification information acquisition unit 111 and the examination history information of the subject patient acquired by the examination history information acquisition unit 112 with a predetermined determination criterion and generates recommended action information including a recommended action for the lesion confirmed in the current examination. The recommended action information output unit 113 outputs the generated recommended action information to the endoscope system 20.

The predetermined determination criterion is created based on accumulated data of past endoscopic examinations accumulated in the examination history information holding unit 121. More specifically, the predetermined determination criterion is created based on examination history information of a reference patient other than the subject patient. The examination history information of the reference patient includes diagnosis information of a lesion confirmed in a past examination and information on actions performed on the diagnosed lesion.

As a predetermined determination criterion, the recommended action information output unit 113 can select the examination history information of a reference patient having examination history information that matches or is similar to the conditions for the examination history information of the subject patient and the lesion identification information of the current examination from among pieces of examination history information of a plurality of reference patients accumulated in the examination history information holding unit 121. As recommended action information, the recommended action information output unit 113 outputs action information corresponding to the lesion identification information of the current examination included in the selected examination history information.

The recommended action information output unit 113 determines the similarity between the examination history information of the reference patient and the examination history information of the subject patient (including the lesion identification information of the current examination), for example, as follows. The recommended action information output unit 113 applies a lesion included in the lesion identification information of the current examination to the qualitative diagnosis of the current examination history information of the subject patient. The action information in the current examination history information is left blank.

The recommended action information output unit 113 extracts examination history information of a reference patient having an examination type that matches the examination type of the subject patient from the pieces of examination history information of a plurality of reference patients. From the extracted examination history information of the reference patient, the recommended action information output unit 113 extracts examination history information of the reference patient for the number of examinations that matches the number of examinations (including the current examination) of the subject patient.

For example, when the examination history information of the reference patient includes examination history for five examinations and when the current examination of the subject patient is the fourth time, the recommended action information output unit 113 extracts examination history information for up to the fourth examination of the reference patient. If the number of examinations of the reference patient is less than the number of examinations of the subject patient, the recommended action information output unit 113 excludes the examination history information of the reference patient. As a reference candidate, the recommended action information output unit 113 uses examination history information of a reference patient that meets the conditions for the examination type and the number of examinations.

The recommended action information output unit 113 extracts examination history information of a reference patient having an examination interval that matches or is similar to the examination interval of the subject patient from among reference candidates. For example, using a similarity score table of examination intervals, the recommended action information output unit 113 sets the similarity score to 1 when the examination interval of the reference patient and the examination interval of the subject patient match and sets the similarity score to a value closer to 0 as the examination interval of the reference patient deviates from the examination interval of the subject patient. The similarity score table of examination intervals is pre-set based on examination pattern identity assessment from an epidemiological point of view. The recommended action information output unit 113 sums up similarity scores for the examination intervals of the respective examinations, and divides the sum by the number of examinations so as to calculate an average similarity score of the examination intervals. The recommended action information output unit 113 extracts examination history information of a reference patient whose average similarity score for examination intervals is a predetermined value (for example, 0.8) or more from among the reference candidates.

The recommended action information output unit 113 extracts examination history information of a reference patient having a qualitative diagnosis that matches or is similar to the qualitative diagnosis of the subject patient from among reference candidates. For example, using a similarity score table of qualitative diagnoses, the recommended action information output unit 113 sets the similarity score to 1 when the qualitative diagnosis of the reference patient and the qualitative diagnosis of the subject patient match and sets the similarity score to a value closer to 0 as the qualitative diagnosis of the reference patient deviates from the qualitative diagnosis of the subject patient. The similarity score table of qualitative diagnoses is pre-set based on the similarity between lesions from an epidemiological point of view. The recommended action information output unit 113 sums up similarity scores for the qualitative diagnoses of the respective examinations, and divides the sum by the number of examinations so as to calculate an average similarity score of the qualitative diagnoses. The recommended action information output unit 113 extracts examination history information of a reference patient whose average similarity score for qualitative diagnoses is a predetermined value (for example, 0.8) or more from among the reference candidates.

The recommended action information output unit 113 extracts the examination history information of the reference patient having action information that matches action information for up to the last examination of the subject patient from the pieces of examination history information of the reference patients that meet the conditions for examination intervals and qualitative diagnosis. As a predetermined determination criterion, the recommended action information output unit 113 selects the examination history information of a reference patient with the highest total score of the average similarity score for examination intervals and the average similarity score of the qualitative diagnosis when there are a plurality of pieces of extracted examination history information of the reference patients. As a predetermined determination criterion, the recommended action information output unit 113 may select the examination history information of a reference patient whose gender is the same and who is close in age from the examination history information of reference patients with the top N total score.

The recommended action information output unit 113 can use a time-axis search when searching for examination history information that matches the conditions from among pieces of examination history information of a plurality of reference patients accumulated in the examination history information holding unit 121. The time-axis search is a search function for extracting search history information of patients that meet a reference point condition in the future direction or in the past direction from a certain reference point.

Further, the predetermined determination criterion may be created based on statistical information of examination history information of a plurality of reference patients. The diagnostic action progress pattern generation unit 114 uses a plurality of pieces of examination history information accumulated in the examination history information holding unit 121 as reference data, and generates a plurality of pieces of diagnostic action progress pattern information by aggregating pieces of examination history information with a matching examination type, a matching or similar examination interval, matching or similar qualitative diagnosis, and matching action information. The diagnostic action progress pattern generation unit 114 accumulates the plurality of pieces of generated diagnostic action progress pattern information in the diagnostic action progress pattern holding unit 122. Each piece of the diagnostic action progress pattern information includes a counted number. The counted number can be considered as the number of cases or the number of patients that followed the progress according to each diagnostic action progress pattern.

The similar range of examination intervals that can be counted as the same diagnostic action progress pattern is pre-set based on examination pattern identity evaluation from an epidemiological point of view. For example, the similarity range of examination intervals is set to X days (for example, 30 days) plus or minus a standard examination interval (1 year, 6 months, 3 months, etc.) for each examination type. The similar range of qualitative diagnoses that can be counted as the same diagnostic action progress pattern is pre-set based on the similarity between lesions from an epidemiological point of view.

As a predetermined determination criterion, the recommended action information output unit 113 can select diagnostic action progress pattern information that matches or is similar to the conditions for the examination history information of the subject patient and the lesion identification information of the current examination from among the plurality of diagnostic action progress pattern information accumulated in the diagnostic action progress pattern holding unit 122. When there is no diagnostic action progress pattern information that matches the conditions for the examination history information of the subject patient and the lesion identification information of the current examination, the recommended action information output unit 113 selects a similar diagnostic action progress pattern information in the same manner as when selecting a predetermined determination criterion from the examination history information of the reference patient described above.

When there are a plurality of pieces of diagnostic action progress pattern information that match the conditions for the examination history information of the subject patient and the lesion identification information of the current examination, the recommended action information output unit 113 selects diagnostic action progress pattern information with the largest number of counts. As recommended action information, the recommended action information output unit 113 outputs action information corresponding to the lesion identification information of the current examination included in the selected diagnostic action progress pattern information.

The recommended action display control unit 115 outputs a control signal to the endoscope processing device 22 that is for superimposing and displaying the recommended action information output by the recommended action information output unit 113 on an endoscopic image of the subject patient being displayed in real time on the display device 23 of the endoscope system 20.

The endoscope processing device 22 displays a guidance frame that surrounds a site of a lesion detected by the image diagnosis device 30 in a superimposed manner on an endoscopic image of the subject patient displayed on the display device 23. The endoscope processing device 22 further displays a guidance display of a recommended action acquired from the endoscope work assistance system 10 in a superimposed manner near the guidance frame that surrounds the site of the lesion.

The report output unit 116 can output an examination report describing matters to be handed over that are based on the recommended action. The examination report is output to and recorded in the examination history information holding unit 121 of the endoscope work assistance system 10 or a linked electronic medical record system (not shown). The examination report will be referred to by the doctor who has performed the current examination or another doctor at the time of subsequent examinations. An examination report is an effective tool for contacting another doctor or reminding the doctor in charge of actions that could not be performed on the day of the examination and should be performed at the next examination. The report output unit 116 may automatically input actions recommended in the current examination but not performed this time as a draft of informative matters on the report input screen.

FIG. 3A to FIG. 3C are diagrams for explaining a flow until a recommended action according to Specific Example 1 is determined. FIG. 3A shows examination history information of a reference patient selected as a predetermined determination criterion according to Specific Example 1. According to the examination history information, four upper endoscopic examinations have been performed in the past at intervals of one year (365 days), and the progress of qualitative diagnosis and action information is as follows: “no abnormal findings” and “no”→“gastric ulcer” and “no”→“fundic gland polyp” and “biopsy”→“early-stage gastric cancer” and “ESD (endoscopic submucosal dissection).” That is, this is a case in which upper endoscopic examinations were performed at intervals of one year, and ESD was performed on the patient with early-stage gastric cancer. Since early-stage gastric cancer that develops through fundic gland polyps tends to progress rapidly, it is preferable to perform ESD, which is an action suitable for widespread and deep-seated cancer.

FIG. 3B shows the examination history information of the subject patient according to Specific Example 1 (the qualitative diagnosis and action information of the current examination have not yet been decided). The examination history information for the past three examinations of the subject patient shown in FIG. 3B matches the examination interval, qualitative diagnosis, and action information for the past three upper endoscopic examinations of the reference patient shown in FIG. 3A.

FIG. 3C shows examination history information after image diagnosis in the current examination of the subject patient according to Specific Example 1. In the current examination, early-stage gastric cancer is detected by image diagnosis by the image diagnosis device 30. The endoscope work assistance system 10 proposes ESD as a recommended action for the current examination based on the examination history information of the reference patient shown in FIG. 3A.

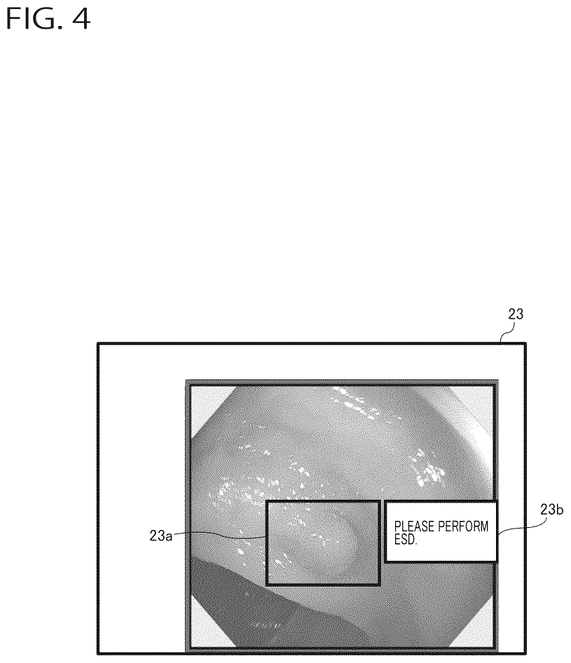

FIG. 4 is a diagram showing an endoscopic image of a subject patient displayed on the display device 23 of the endoscope system 20 according to Specific Example 1. In the endoscopic image displayed on the display device 23, a guidance frame 23a that surrounds a site of a lesion diagnosed by the image diagnosis device 30 is superimposed, and a guidance display 23b for a recommended action “Please perform ESD.” is superimposed near the guidance frame 23a.

FIG. 5A to FIG. 5C are diagrams for explaining a flow until a recommended action according to Specific Example 2 is determined. FIG. 5A shows examination history information of a reference patient selected as a predetermined determination criterion according to Specific Example 2. According to the examination history information, four upper endoscopic examinations have been performed in the past at intervals of one year (365 days), and the progress of qualitative diagnosis and action information is as follows: “no abnormal findings” and “no”→“gastric ulcer” and “no”→“adenoma type 01” and “biopsy”→“early-stage gastric cancer” and “EMR (endoscopic mucosal resection).” That is, this is a case in which upper endoscopic examinations were performed at intervals of one year, and EMR was performed on the patient with early-stage gastric cancer. Since early-stage gastric cancer that develops through adenoma type 01 tends to progress slowly, it is likely that the cancer can be removed with EMR, which is an action suitable for small lesions.

FIG. 5B shows the examination history information of the subject patient according to Specific Example 2 (the qualitative diagnosis and action information of the current examination have not yet been decided). The examination history information for the past three examinations of the subject patient shown in FIG. 5B matches the examination interval, qualitative diagnosis, and action information for the past three upper endoscopic examinations of the reference patient shown in FIG. 5A.

FIG. 5C shows examination history information after image diagnosis in the current examination of the subject patient according to Specific Example 2. In the current examination, early-stage gastric cancer is detected by image diagnosis by the image diagnosis device 30. The endoscope work assistance system 10 proposes EMR as a recommended action for the current examination based on the examination history information of the reference patient shown in FIG. 5A. Since the progress pattern is different from that in the examination history information of the reference patient according to Specific Example 1 shown in FIG. 3A, an action different from that in Specific Example 1 is proposed.

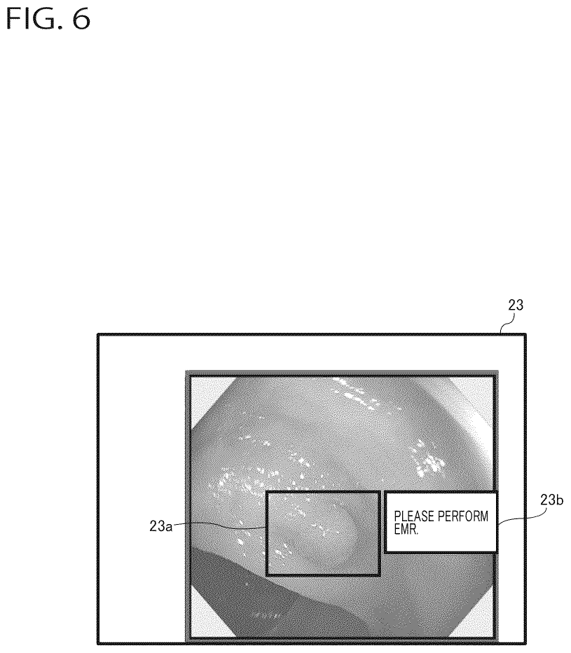

FIG. 6 is a diagram showing an endoscopic image of a subject patient displayed on the display device 23 of the endoscope system 20 according to Specific Example 2. In the endoscopic image displayed on the display device 23, a guidance frame 23a that surrounds a site of a lesion diagnosed by the image diagnosis device 30 is superimposed, and a guidance display 23b for a recommended action “Please perform EMR.” is superimposed near the guidance frame 23a.

FIG. 7A to FIG. 7C are diagrams for explaining a flow until a recommended action according to Specific Example 3 is determined. FIG. 7A shows diagnostic action progress pattern information A that serves as a candidate for a predetermined determination criterion according to Specific Example 3. The diagnostic action progress pattern information A is information that aggregates examination history information from four upper endoscopic examinations performed in the past at intervals of one year (365 days) where a progress pattern of qualitative diagnosis and action information follows a course “no abnormal findings” and “no”→“gastric ulcer” and “biopsy”→“fundic gland polyp” and “polypectomy”→“adenoma 0II-b” and “ESD.” The number of cases that followed this progress pattern is 100.

FIG. 7B shows diagnostic action progress pattern information B that serves as a candidate for a predetermined determination criterion according to Specific Example 3. The diagnostic action progress pattern information B is information that aggregates examination history information from four upper endoscopic examinations performed in the past at intervals of one year (365 days) where a progress pattern of qualitative diagnosis and action information follows a course “no abnormal findings” and “no”→“gastric ulcer” and “biopsy”→“fundic gland polyp” and “polypectomy”→“adenoma 0II-b” and “adenoma ablation.” The number of cases that followed this progress pattern is 300.

FIG. 7C shows examination history information after image diagnosis in the current examination of the subject patient according to Specific Example 3. In the current examination, adenoma 0II-b is detected by image diagnosis by the image diagnosis device 30. When there are a plurality of pieces of diagnostic action progress pattern information (in Specific Example 3, diagnostic action progress pattern information A and diagnostic action progress pattern information B) that match the examination history information of the subject patient and the lesion identification information of the current examination (in Specific Example 3, adenoma 0II-b), the endoscope work assistance system 10 selects the diagnostic action progress pattern information B with a larger number of cases as the predetermined determination criterion. The endoscope work assistance system 10 proposes adenoma ablation as the recommended action for the current examination.

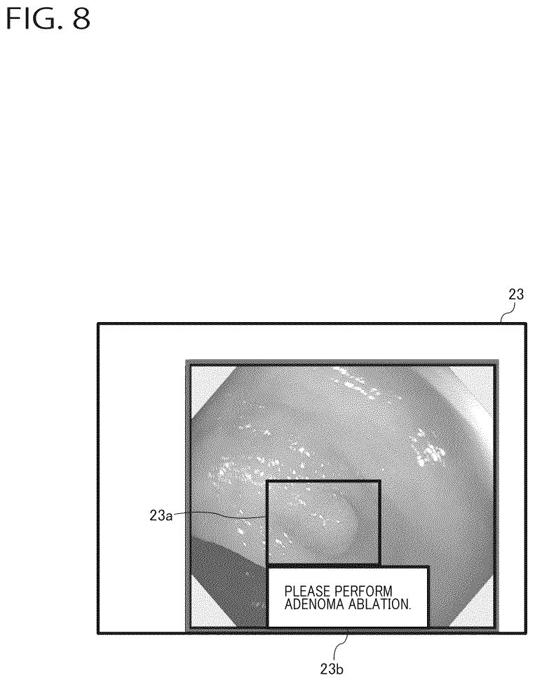

FIG. 8 is a diagram showing an endoscopic image of a subject patient displayed on the display device 23 of the endoscope system 20 according to Specific Example 3. In the endoscopic image displayed on the display device 23, a guidance frame 23a that surrounds a site of a lesion diagnosed by the image diagnosis device 30 is superimposed, and a guidance display 23b for a recommended action “Please perform adenoma ablation.” is superimposed near the guidance frame 23a.

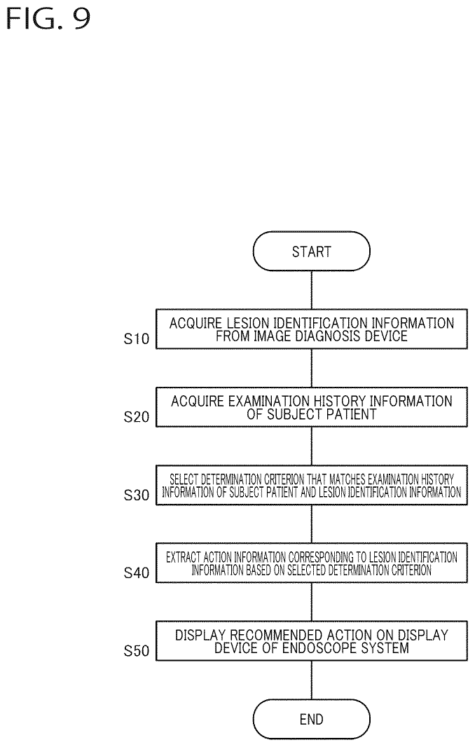

FIG. 9 is a flowchart showing the basic operation of the endoscope work assistance system 10 according to the embodiment. The lesion identification information acquisition unit 111 acquires lesion identification information for the current examination of the subject patient from the image diagnosis device 30 (S10). The examination history information acquisition unit 112 acquires examination history information of the subject patient from the examination history information holding unit 121 (S20). The recommended action information output unit 113 selects a determination criterion that matches the conditions for the examination history information of the subject patient and the lesion identification information of the current examination (S30). The recommended action information output unit 113 extracts action information corresponding to the lesion identification information of the current examination based on the selected determination criterion (S40). The recommended action display control unit 115 superimposes the extracted action information on an endoscopic image displayed on the display device 23 as a recommended action (S50).

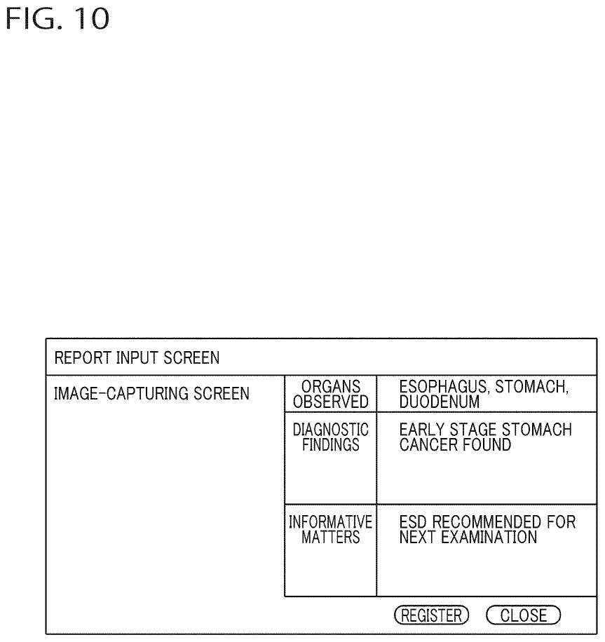

FIG. 10 shows an example of a report input screen. In addition to the diagnostic findings of the current examination, as an informative matter, the doctor can enter an action that was recommended by the endoscope work assistance system 10 but could not be performed in the current examination as a matter to be handed over.

As explained above, according to the present embodiment, by presenting a recommended action that is based on the examination history information of the subject patient and the lesion identification information of the current examination to the doctor performing an endoscopic examination, it is possible to assist in making decisions regarding if there are actions for the lesion and determining the details of the action. Some conventional medical diagnosis assistance systems also display inferred findings on an endoscope monitor. However, the decision as to if there are actions for the lesion and determining the details of the action based on the displayed finding information depends on the experience of the doctor. If the doctor makes an error in judgment, proper actions may not be performed. According to the present embodiment, the recommended action output from the endoscopic service assistance system 10 is superimposed on the display device 23 of the endoscope system 20, thereby making it possible to prevent omission of an action that should be performed.

Described above is an explanation based on the embodiments of the present disclosure. These embodiments are intended to be illustrative only, and it will be obvious to those skilled in the art that various modifications to constituting elements and processes could be developed and that such modifications are also within the scope of the present disclosure.

In the above embodiments, as lesion identification information of the current examination of the subject patient, lesion identification information is used that includes the result of identifying a lesion confirmed by image recognition of an endoscopic image performed by the image diagnosis device 30. In this regard, lesion identification information may be used that includes the result of identifying a lesion confirmed by the doctor in the current examination.

In the above embodiments, the image diagnosis device 30 may perform part or all of the processes performed by the processing unit 11 of the endoscope work assistance system 10. Further, the endoscope work assistance system 10 and the image diagnosis device 30 may be constructed in one server.

Further, part or all of the processes performed by the processing unit 11 of the endoscope work assistance system 10 may be performed on a cloud server. For example, the process of the diagnostic action progress pattern generation unit 114 may be performed on a cloud server. In that case, the diagnostic action progress pattern generation unit 114 can collect examination history information of a plurality of patients from a plurality of endoscope work assistance systems 10 installed in the endoscope departments of a plurality of medical facilities. The diagnostic action progress pattern generation unit 114 generates a plurality of pieces of diagnostic action progress pattern information based on a large number of collected pieces of examination history information. The diagnostic action progress pattern generation unit 114 provides the plurality of pieces of generated diagnostic action progress pattern information to each endoscope work assistance system 10 installed in each medical facility. In this case, more reliable diagnostic action progress pattern information can be generated.

What is claimed is:

1. A medical assistance system comprising:
at least one processor comprising hardware, wherein
wherein the at least one processor is configured to:
acquire lesion identification information including the result of identifying a lesion confirmed in a current endoscopic examination of a patient being examined;
acquire examination history information including an examination type and, an examination interval, and the result of identifying the lesion from a diagnosis history for a past endoscopic examination of the patient being examined; and
compare the lesion identification information and the examination history information with a predetermined determination criterion that is created based on examination history information of a reference patient other than the patient being examined and that includes the examination type, the examination interval, and the result of identifying the lesion as conditions and output recommended action information including a recommended action for the result of identifying the lesion confirmed in the current endoscopic examination.

2. The medical assistance system according to claim 1, wherein
the predetermined determination criterion is created based on accumulated data of past endoscopic examinations.

3. The medical assistance system according to claim 1, wherein
the predetermined determination criterion is created based on examination history information of a reference patient other than the patient being examined, and
the examination history information of the reference patient includes diagnosis information of a lesion confirmed in a past examination and information on an action performed on the diagnosed lesion.

4. The medical assistance system according to claim 1, further including:
a storage unit that stores examination history information of a plurality of reference patients, wherein
the at least one processor selects examination history information of a reference patient having examination history information that matches or is similar to conditions for the examination history information of the patient being examined and the lesion identification information of the current endoscopic examination from among pieces of examination history information of the plurality of reference patients, and outputs, as the recommended action information, action information corresponding to the lesion identification information of the current endoscopic examination included in the selected examination history information of the reference patient.

5. The medical assistance system according to claim 1, wherein
the predetermined determination criterion is created based on statistical information of examination history information of the plurality of reference patients.

6. The medical assistance system according to claim 5, further including:
a storage unit that stores a plurality of pieces of diagnostic action progress pattern information aggregated based on the examination history information of the plurality of reference patients, wherein
the at least one processor selects diagnostic action progress pattern information that matches or is similar to conditions for the examination history information of the patient being examined and the lesion identification information of the current endoscopic examination from among the plurality of pieces of diagnostic action progress pattern information, and outputs, as the recommended action information, action information corresponding to the lesion identification information of the current endoscopic examination included in the selected diagnostic action progress pattern information.

7. The medical assistance system according to claim 6, wherein
the plurality of pieces of diagnostic action progress pattern information include a counted number of patients, and
when there are a plurality of pieces of diagnostic action progress pattern information that match the conditions for the examination history information of the patient being examined and the lesion identification information of the current endoscopic examination, the at least one processor selects diagnostic action progress pattern information with the largest number of counted patients.

8. The medical assistance system according to claim 4, wherein
the examination history information of the plurality of reference patients includes examination interval information, and
the at least one processor selects examination history information of a reference patient having diagnosis information, examination interval information, and action information of a past endoscopic examination that matches or is similar to conditions for diagnosis information, examination interval information, and action information of a past endoscopic examination of the patient being examined and the lesion identification information of the current endoscopic examination from among the pieces of examination history information of the plurality of reference patients.

9. The medical assistance system according to claim 1, wherein
the at least one processor acquires lesion identification information including the result of identifying a lesion detected based on image recognition by an image diagnosis device in the endoscopic examination of the patient being examined.

10. The medical assistance system according to claim 1, wherein
the at least one processor displays the recommended action information in a superimposed manner on an endoscopic image of the patient being examined that is displayed in real time on a monitor.

11. The medical assistance system according to claim 10, wherein
the at least one processor is configured to:
output an examination report describing a matter to be handed over that is based on the recommended action.

12. A medical assistance method comprising:
acquiring lesion identification information including the result of identifying a lesion confirmed in a current endoscopic examination of a patient being examined;
acquiring examination history information including an examination type and, an examination interval, and the result of identifying the lesion from a diagnosis history for a past endoscopic examination of the patient being examined; and
comparing the lesion identification information and the examination history information with a predetermined determination criterion that is created based on examination history information of a reference patient other than the patient being examined and that includes the examination type, the examination interval, and the result of identifying the lesion as conditions and outputting recommended action information including a recommended action for the result of identifying the lesion confirmed in the current endoscopic examination.

13. A storage medium having embodied thereon a program comprising computer-implemented modules including:
a module that acquires lesion identification information including the result of identifying a lesion confirmed in a current endoscopic examination of a patient being examined;
a module that acquires examination history information including an examination type and, an examination interval, and the result of identifying the lesion from a diagnosis history for a past endoscopic examination of the patient being examined; and
a module that compares the lesion identification information and the examination history information with a predetermined determination criterion that is created based on examination history information of a reference patient other than the patient being examined and that includes the examination type, the examination interval, and the result of identifying the lesion as conditions and outputs recommended action information including a recommended action for the result of identifying the lesion confirmed in the current endoscopic examination.

14. A medical control device including at least one processor comprising hardware, wherein
the at least one processor is configured to:
acquire lesion identification information including the result of identifying a lesion confirmed in a current endoscopic examination of a patient being examined;
acquire examination history information including an examination type and, an examination interval, and the result of identifying the lesion from a diagnosis history for a past endoscopic examination of the patient being examined; and
compare the lesion identification information and the examination history information with a predetermined determination criterion that is created based on examination history information of a reference patient other than the patient being examined and that includes the examination type, the examination interval, and the result of identifying the lesion as conditions and output recommended action information including a recommended action for the result of identifying the lesion confirmed in the current endoscopic examination.

15. The medical control device according to claim 14, wherein
the predetermined determination criterion is created based on accumulated data of past endoscopic examinations.

16. The medical control device according to claim 14, wherein
the predetermined determination criterion is created based on examination history information of a reference patient other than the patient being examined, and
the examination history information of the reference patient includes diagnosis information of a lesion confirmed in a past examination and information on an action performed on the diagnosed lesion.

17. The medical control device according to claim 14, wherein
the predetermined determination criterion is created based on statistical information of examination history information of the plurality of reference patients.

18. The medical control device according to claim 14, further including:
a storage unit that stores a plurality of pieces of diagnostic action progress pattern information aggregated based on the examination history information of the plurality of reference patients, wherein
the at least one processor selects diagnostic action progress pattern information that matches or is similar to conditions for the examination history information of the patient being examined and the lesion identification information of the current endoscopic examination from among the plurality of pieces of diagnostic action progress pattern information, and outputs, as the recommended action information, action information corresponding to the lesion identification information of the current endoscopic examination included in the selected diagnostic action progress pattern information.

19. The medical control device according to claim 18, wherein
the plurality of pieces of diagnostic action progress pattern information include a counted number of patients, and
when there are a plurality of pieces of diagnostic action progress pattern information that match the conditions for the examination history information of the patient being examined and the lesion identification information of the current endoscopic examination, the at least one processor selects diagnostic action progress pattern information with the largest number of counted patients.

20. The medical control device according to claim 14, wherein
the examination history information of the plurality of reference patients includes examination interval information, and
the at least one processor selects examination history information of a reference patient having diagnosis information, examination interval information, and action information of a past endoscopic examination that matches or is similar to conditions for diagnosis information, examination interval information, and action information of a past endoscopic examination of the patient being examined and the lesion identification information of the current endoscopic examination from among the pieces of examination history information of the plurality of reference patients.
